(19) United States
(12) Patent Application Publication
(10) Pub. No.: US 2022/0175589 A1
(43) Pub. Date: Jun. 9, 2022

(54) FOLDED ABSORBENT ARTICLE AND INDIVIDUAL ABSORBENT ARTICLE PACKAGE

(71) Applicant: LIVEDO CORPORATION, Ehime (JP)

(72) Inventor: Riho YOSHIDA, Tokushima (JP)

(73) Assignee: LIVEDO CORPORATION, Ehime (JP)

(21) Appl. No.: 17/437,293

(22) PCT Filed: Feb. 13, 2020
(86) PCT No.: PCT/JP2020/005559

§ 371 Date: Sep. 8, 2021

(30) Foreign Application Priority Data
Mar. 11, 2019 (JP) 2019-044010

(51) Int. Cl. A61F 13/47; C08J 5/18; A61F 13/56

(52) U.S. Cl. CPC A61F 13/4704; A61F 13/5611; A61F 13/56; C08J 5/18

(57) ABSTRACT

A folded absorbent article, wherein: the absorbent article (1) comprises a top sheet (2), a back sheet (3) and an absorbent body (4) provided therebetween; the absorbent article (1) is folded in three in the front-rear direction with a skin facing side inside so that a front part (11) and a rear part (13) overlap with a middle part (12); the absorbent body (4) is provided so as to extend from the middle part (12) to the front part (11) and the rear part (13); and an anti-slipping member (7) is provided in the middle part (12) so as to overlap with the absorbent body (4) and attaching-fixing members (8) made of a hook member or an adhesive layer are provided in the front part (11) and the rear part (13) on a non-skin facing side of the back sheet (4).

### TECHNICAL FIELD

The present invention relates to a folded absorbent article which is formed by deployably folding an absorbent article, such as an urine pad, an incontinence pad and a sanitary napkin, that can be attached to underwear such as pants or shorts, and relates to an individual package of the absorbent article.

### BACKGROUND ART

Conventionally, an absorbent article provided with a hook member or an adhesive layer on a non-skin facing side thereof, that is used by being attached to a skin facing side of underwear such as pants and shorts with the hook member or the adhesive layer, is known (for example, Patent Literatures 1 and 2). Further, an absorbent article provided with an anti-slipping layer on a non-skin facing side thereof is also known (for example, Patent Literature 3).

### CITATION LIST

### Patent Literature

### Patent Literature 1

Japanese Unexamined Laid-open Patent Application Publication No. H11-506365

### Patent Literature 2

Japanese Unexamined Laid-open Patent Application Publication No. 2003-210524

### Patent Literature 3

Japanese Unexamined Laid-open Patent Application Publication No. 2008-11932

### SUMMARY OF INVENTION

### Technical Problem

In the case where an absorbent article is attached to a skin facing side of underwear such as pants or shorts to be used, it is desired that the absorbent article is properly attached to an intended position on the skin facing side of the underwear. The present invention has been made in view of the above circumstances, and an object of the present invention is to provide a folded absorbent article that can be easily attached to an intended position of underwear in attaching the absorbent article to a skin facing side of underwear to use and an individual absorbent article package in which the absorbent article is wrapped with a packaging sheet.

### Solution to Problem

The disposable diaper of the present invention which solves the above problems is a folded absorbent article, wherein; the absorbent article has a width direction and a front-rear direction, and comprises a top sheet, a back sheet and an absorbent body provided therebetween; the absorbent article has a front part, a rear part and a middle part positioned therebetween in the front-rear direction, and is folded in three in the front-rear direction with a skin facing side inside so that the front part and the rear part overlap with the middle part; the absorbent body is provided so as to extend from the middle part to the front part and the rear part; an anti-slipping member is provided on a non-skin facing side of the back sheet in the middle part so as to overlap with the absorbent body; and attaching-fixing members made of a hook member or an adhesive layer are provided on the non-skin facing side of the back sheet in the front part and the rear part.

Since the folded absorbent article of the present invention is provided with the anti-slipping member and the attaching-fixing member on the non-skin facing side of the back sheet as described above, it becomes easy to attach the absorbent article to an appropriate position of underwear. That is, in attaching the absorbent article to underwear, the middle part of the absorbent article is moved along a skin facing side of the underwear without completely deploying the absorbent article in a plane, whereby the absorbent article can be set at an appropriate position on the skin facing side of the underwear. After the position of setting the absorbent article is determined, the absorbent article is deployed in a plane with the middle part held on the underwear utilizing the anti-slipping member provided in the middle part, the front part and the rear part are pressed against the underwear to fastening the attaching-fixing member to the underwear, whereby it becomes easy to attach the absorbent article to an intended position appropriately on the skin facing side of the underwear without being displaced.

It is preferable that the anti-slipping member is not provided on a front side of the attaching-fixing member in the front part and on a rear side of the attaching-fixing member in the rear part. By providing the anti-slipping member in this manner, front and rear end portions of the absorbent article are formed relatively flexible, and fittability of the absorbent article is easily enhanced in these portions.

It is preferable that the attaching-fixing member is provided so as to overlap with a front end of the absorbent body or on a front side of a front end of the absorbent body in the front part, and the attaching-fixing member is provided so as to overlap with a rear end of the absorbent body or on a rear side of a rear end of the absorbent body in the rear part. By providing the attaching-fixing member in this manner, the absorbent article can be stably fixed to a skin facing side of underwear.

It is preferable that a fiber sheet is provided between the absorbent body and the back sheet, the fiber sheet has a larger mass per unit area than the top sheet and the back sheet, and the fiber sheet is provided so as to extend to a front side of a front end of the absorbent body and extend to a rear side of a rear end of the absorbent body. As a result, rigidity of the front and rear end portions of the absorbent article where the absorbent body does not exist can be increased. Therefore, the front and rear end portions of the absorbent article can be prevented from bending, in attaching the absorbent article to underwear, and the absorbent article can be stably attached to a skin facing side of underwear.

It is preferable that the anti-slipping member is provided only in the middle part. By providing the anti-slipping member only in the middle part, the front part and the rear part of the absorbent article are formed relatively flexible, and fittability of the absorbent article is easily enhanced in these parts. In addition, by providing the anti-slipping member only in the middle part, the folded absorbent article can be formed thin without being bulky.

It is preferable that a plurality of the anti-slipping members are aligned in the front-rear direction. By providing a plurality of the anti-slipping member so as to align in the front-rear direction, shear stress generated in one anti-slipping member is not directly transmitted to another anti-slipping member in the state where the absorbent article is attached to underwear. Therefore, the position where the absorbent article is attached to underwear can be prevented from displacing and the absorbent article can be prevented from being distorted over a wide range. Further, by arranging a plurality of the anti-slipping members in the front-rear direction, it becomes easy to bend the absorbent article in the front-rear direction, and fittability of the absorbent article to a wearer's crotch can be improved.

It is preferable that the absorbent article is folded back at a first fold line and a second fold line each extending in the width direction, thereby being folded in three in the front-rear direction, and the anti-slipping members are provided in a region within 10 mm in the front-rear direction from the first fold line and a region within 10 mm in the front-rear direction from the second fold line. At the first fold line and its vicinity and at the second fold line and its vicinity, the absorbent article is formed to protrude toward the non-skin facing side in the state where the absorbent article is deployed in a plane, resulting in being likely to strongly contact with underwear at these portions. Therefore, by providing the anti-slipping member at such a part, it becomes easy to maintain the middle part of the absorbent article to be held at the determined position on the skin facing side of the underwear in deploying the folding of the absorbent article to spread in a plane.

It is preferable that the anti-slipping member is formed of a resin layer. Thereby, anti-slipping effect of the anti-slipping member is likely to be suitably exerted.

The present invention also provides an individual absorbent article package comprising a packaging sheet and the above-described folded absorbent article wrapped with the packaging sheet, wherein the packaging sheet is overlaid on a non-skin facing side of the absorbent article, detachably attached to the attaching-fixing member, and folded in three in the front-rear direction together with the absorbent article. In the individual absorbent article package of the present invention, the absorbent article can be attached to a skin facing side of underwear by deploying the folding of the individual package and peeling off the packaging sheet from the absorbent article. At this time, a non-skin facing side of the middle part of the absorbent article is set at an appropriate position on a skin facing side of underwear in the state where the absorbent article is not completely deployed in a plane, the absorbent article is deployed in a plane with the middle part of the absorbent article held on the underwear, and the front part and the rear part are pressed against the underwear, whereby it becomes easy to attach the absorbent article to an intended position on the skin facing side of the underwear.

### Advantageous Effects of Invention

According to the folded absorbent article, in attaching the absorbent article to underwear to use, the middle part of the absorbent article is moved along a skin facing side of the underwear without completely deploying the absorbent article in a plane, whereby the absorbent article can be set at an appropriate position on the skin facing side of the underwear. After the position of setting the absorbent article is determined, the absorbent article is deployed in a plane with the middle part held on the underwear utilizing the anti-slipping member provided in the middle part, the front part and the rear part are pressed against the underwear to fastening the attaching-fixing member to the underwear, whereby the absorbent article can be easily attached to an intended position appropriately on the skin facing side of the underwear without being displaced.

### BRIEF DESCRIPTION OF THE DRAWINGS

FIG. 1 shows an example of an absorbent article of the present invention, and shows a plan view of the absorbent article in a deployed state as viewed from a skin facing side thereof.

FIG. 2 shows a plan view of the absorbent article shown in FIG. 1 as viewed from a non-skin facing side thereof.

FIG. 3 shows a cross-sectional view taken along a line of the absorbent article shown in FIGS. 1 and 2.

FIG. 4A and FIG. 4B show an example of a method for forming an individual absorbent article package, and FIG. 4B shows a plan view of the individual absorbent article package.

### DESCRIPTION OF THE PREFERRED EMBODIMENTS

The present invention relates to a folded absorbent article, and particularly relates to a folded absorbent article which is formed by folding an absorbent article, such as an urine pad, an incontinence pad and a sanitary napkin, that can be attached to underwear such as pants or shorts to use.

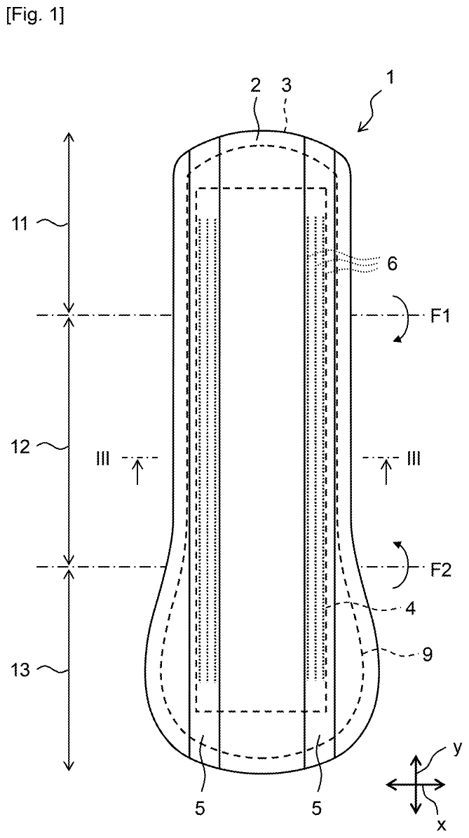

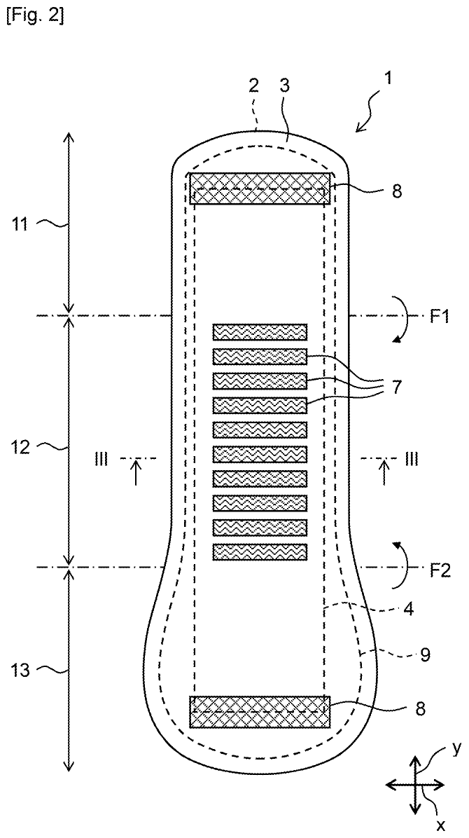

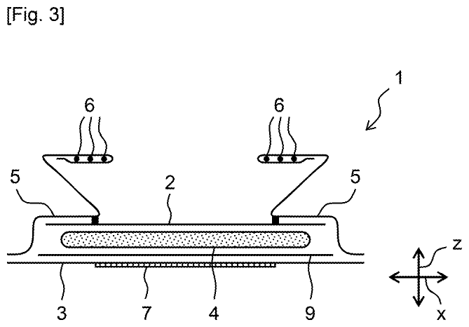

First, a basic constitution of an absorbent article used in the present invention is explained with reference to the drawings. FIG. 1 shows a plan view of the absorbent article (an incontinence pad) in a deployed planar state as viewed from a skin facing side thereof, FIG. 2 shows a plan view of the absorbent article shown in FIG. 1 as viewed from a non-skin facing side thereof, and FIG. 3 shows a cross-sectional view taken along a line of the absorbent article shown in FIGS. 1 and 2. In the drawings of the present application, an arrow x represents a width direction, an arrow y represents a front-rear direction, and a direction perpendicular to a plane formed by the arrows x and y represents a thickness direction z. In FIGS. 1 and 2, an upper side of the drawing corresponds to a front side of the absorbent article, and a lower side of the drawing corresponds to a rear side of the absorbent article. However, the present invention is not limited to the embodiment shown in the drawings.

An absorbent article 1 comprises a top sheet 2, a back sheet 3 and an absorbent body 4 provided therebetween. The top sheet 2 is disposed on a skin facing side of the absorbent body 4, and the back sheet 3 is disposed on a non-skin facing side of the absorbent body 4. Excrement that has passed through the top sheet 2 is stored by the absorbent body 4. The back sheet 3 prevents excrement from leaking to the outside.

The absorbent article 1 has a front-rear direction y and a width direction x. The front-rear direction y means a direction extending in a front-rear direction at a crotch of a wearer when the absorbent article is worn. The width direction x means a direction orthogonal to the front-rear direction y on the same plane as the absorbent article, and corresponds to a left-right direction of a wearer when the absorbent article is worn. In addition, a skin facing side means a side facing a wearer's skin and a non-skin facing side means an opposite side of that when a wearer wears the absorbent article.

The top sheet 2 is disposed on a skin facing side of the absorbent body 4 and is provided so as to face a wearer's skin when the absorbent article is worn. The top sheet 2 is preferably liquid permeable. As the top sheet 2, a nonwoven fabric made from hydrophilic fibers such as cellulose, rayon and cotton; a nonwoven fabric which is formed from hydrophobic fibers such as polyolefin (e.g., polypropylene and polyethylene), polyester (e.g., PET) and polyamide (e.g., nylon), and in which the hydrophobic fibers are hydrophilized with a surfactant on the surface thereof; or the like can be used, for example. As the top sheet 2, a woven fabric, a knitted fabric, a plastic film having holes may be also used.

The back sheet 3 is disposed on a non-skin facing side of the absorbent body 4 and is preferably liquid impermeable. As the back sheet 3, a nonwoven fabric made from hydrophobic fibers such as polyolefin (e.g., polypropylene and polyethylene), polyester (e.g., PET) and polyamide (e.g., nylon); a plastic film; or the like can be used, for example. As the back sheet 3, a laminate of a nonwoven fabric and a plastic film may be used. A non-skin facing side of the back sheet 3 serves as an underwear facing surface when the absorbent article is attached to underwear such as pants or shorts.

The absorbent body 4 is not particularly limited as long as it contains an absorbent material that is able to absorb excrement such as urine. As the absorbent body 4, a shaped product of an absorbent material, which is formed into a certain shape, may be used, or the shaped product wrapped with a cover sheet such as paper (e.g., tissue paper and thin paper) and a liquid-permeable nonwoven fabric may be used. Examples of the absorbent material include, for example, a hydrophilic fiber such as a cellulose fiber, and an absorbent polymer such as a polyacrylic absorbent polymer, a polyasparaginic absorbent polymer, a cellulosic absorbent polymer and a stark-acrylonitrile absorbent polymer. The absorbent material may include a thermal fusion fiber such as a polyolefin (e.g., polyethylene and polypropylene) fiber, a polyester (e.g., PET) fiber and a polyamide fiber. These thermal fusion fibers may be hydrophilized with a surfactant or the like to increase affinity with urine or the like.

The absorbent material preferably includes a hydrophilic fiber in view of increasing absorption rate of urine or the like. In addition, in view of enhancing absorption capacity, the absorbent material preferably includes an absorbent polymer. Therefore, the absorbent body 4 preferably contains both a hydrophilic fiber (especially a pulp fiber) and an absorbent polymer. In this case, the absorbent material to be used is preferably formed by mixing an absorbent polymer with a hydrophilic fiber assembly, or dispersing an absorbent polymer on a hydrophilic fiber assembly, for example.

The absorbent body 4 may be a sheet-like absorbent body. Examples of the sheet-like absorbent body include that configured to contain an absorbent polymer but not contain a pulp fiber between nonwoven fabrics. Since the thus formed sheet-like absorbent body contains an absorbent polymer between nonwoven fabrics, a high absorption capacity can be realized. Further, since the sheet-like absorbent body does not contain a pulp fiber between the nonwoven fabrics, it can be formed thin without being bulky.

A shape (planar shape) of the absorbent body 4 is not particularly limited. The shape of the absorbent body 4 may be determined as appropriate according to application, and examples of that include a substantially rectangular shape, an hourglass shape, a center nipped-in gourd shape, a battledore shape and others.

It is preferable that the absorbent article 1 is provided with side sheets 5 extending in the front-rear direction y on both sides of the top sheet 2 in the width direction x. The side sheets 5 are joined to both sides of the top sheet 2 in the width direction x. In FIGS. 1 and 3, three rising elastic members 6 are disposed on each of the side sheets 5 provided on one side and the other side in the width direction x. In using the absorbent article, an inner portion of the side sheet 5 rises toward a wearer's skin due to contraction force of the rising elastic member 6 to form a rising flap, thereby preventing lateral leakage of excrement such as urine. The side sheet 5 can be made of a liquid-impermeable plastic film, a liquid-impermeable nonwoven fabric or the like, and a sheet material that can be used for the back sheet 3 can be employed.

In the drawings, a fiber sheet 9 is provided between the absorbent body 4 and the back sheet 3, and the fiber sheet 9 can improve shape-retaining property of the absorbent article 1. The fiber sheet 9 can be made of crepe paper or a nonwoven fabric.

In the case where a nonwoven fabric is used for the top sheet 2, the back sheet 3 or the side sheet 5, a spunbonded nonwoven fabric, an air-through nonwoven fabric, a point-bonded nonwoven fabric, a meltblown nonwoven fabric, an airlaid nonwoven fabric, an SMS nonwoven fabric or the like is preferably used as the nonwoven fabric.

The absorbent article 1 is folded in three in the front-rear direction y with a skin facing side inside so that a front part 11 and a rear part 13 in the front-rear direction y overlap with a middle part 12 positioned therebetween. That is, the absorbent article 1 is folded back at a first fold F1 and a second fold F2 each extending in the width direction x, thereby being folded in three in the front-rear direction y. The front part 11 and the middle part 12 are sectioned by the first fold line F1, and the middle part 12 and the rear part 13 are sectioned by the second fold line F2. The absorbent body 4 is provided so as to extend from the middle part 12 to the front part 11 and the rear part 13.

On a non-skin facing side of the back sheet 3, an anti-slipping member 7 is provided so as to overlap with the absorbent body 4 in the middle part 12 of the absorbent article 1, and attaching-fixing members 8 made of a hook member or an adhesive layer are provided in the front part 11 and the rear part 13. The anti-slipping member 7 is provided for preventing the absorbent article 1 from displacing with respect to underwear in a state of not being fixed to the underwear in placing the absorbent article 1 on a skin facing side of the underwear such as pants and shorts. Meanwhile, the attaching-fixing member 8 is provided for fixing the absorbent article 1 to a skin facing side of underwear. Since the absorbent article 1 is provided with the anti-slipping member 7 and the attaching-fixing member 8 on the non-skin facing side of the back sheet 3 in this manner, it becomes easy to attach the absorbent article 1 to an appropriate position of underwear. That is, in attaching the absorbent article 1 to underwear, the middle part 12 of the absorbent article 1 is moved along the skin facing side of the underwear without completely deploying the absorbent article 1 in a plane, whereby the absorbent article 1 can be set at an appropriate position on the skin facing side of the underwear. After the position of setting the absorbent article 1 is determined, the absorbent article 1 is deployed in a plane with the middle part 12 held on the underwear, the front part 11 and the rear part 13 are pressed against the underwear to fastening the attaching-fixing member 8 to the underwear, whereby it becomes easy to attach the absorbent article 1 to an intended position appropriately on the skin facing side of the underwear without being displaced

The anti-slipping member 7 is provided at least in the middle part 12, and may also be provided in the front part 11 and the rear part 13. In the case where the anti-slipping member 7 is provided in the front part 11 and the rear part 13, it is preferable that the anti-slipping member 7 is provided so as to overlap with the absorbent body 4 in the front part 11 and the rear part 13. Meanwhile, the attaching-fixing member 8 is preferably provided only in the front part 11 and the rear part 13 and is not provide in the middle part 12. The anti-slipping member 7 is preferably not provided on a front side of the attaching-fixing member 8 in the front part 11 and on a rear side of the attaching-fixing member 8 in the rear part 13. The anti-slipping members 7 provided on the front side of the attaching-fixing member 8 in the front part 11 and on the rear side of the attaching-fixing member 8 in the rear part 13 do not contribute to set the position of the absorbent article 1 in attaching the absorbent article 1 to underwear. Rather, when the anti-slipping member 7 is not provided at such a position, front and rear end portions of the absorbent article 1 are formed relatively flexible, and fittability of the absorbent article 1 is easily enhanced in these portions.

It is preferable that the anti-slipping member 7 is provided at least on the first fold line F1 or its vicinity and on the second fold line F2 or its vicinity. Specifically, it is preferable that the anti-slipping member 7 is provided in a region within 10 mm in the front-rear direction y from the first fold line F1 and in a region within 10 mm in the front-rear direction y from the second fold F2. At the first fold line F1 and its vicinity and at the second fold line F2 and its vicinity, the absorbent article 1 is formed to protrude toward the non-skin facing side in the state where the absorbent article 1 is deployed in a plane, resulting in being likely to strongly contact with underwear at these portions. Therefore, by providing the anti-slipping member 7 at such a portion, it becomes easy to maintain the middle part 12 of the absorbent article 1 to be held at the determined position on the skin facing side of the underwear in deploying the folding of the absorbent article 1 to spread in a plane. The anti-slipping member 7 is more preferably provided in a region within 5 mm, even more preferably provided in a region within 3 mm in the front-rear direction y from the first or second folds F1, F2. The exact position of the first fold line F1 can be determined by marking an overlapping position of the front part 11 and the middle part 12 in the state where the absorbent article 1 is folded in three in the front-rear direction y, and finding a midpoint of the marked position in the front-rear direction y. The exact position of the second fold line F2 can also be determined by the similar method.

The anti-slipping member 7 may be provided only in the middle part 12, and not be provided in the front part 11 and the rear part 13. By providing the anti-slipping member 7 only in the middle part 12, the front part 11 and the rear part 13 of the absorbent article 1 are formed relatively flexible, and fittability of the absorbent article 1 is easily enhanced in these parts. In addition, by providing the anti-slipping member 7 only in the middle part 12, folding back the absorbent article 1 at the first fold F1 and the second fold line F2 is facilitated, and the folded absorbent article 1 can be formed thin without being bulky.

It is preferable that a plurality of the anti-slipping members 7 are aligned in the front-rear direction y. The anti-slipping member 7 can be expected to have anti-slipping effect (particularly, anti-slipping effect in the middle part 12) of the absorbent article 1 in attaching the absorbent article 1 to a skin surface side of underwear, and by providing a plurality of the anti-slipping member 7 so as to align in the front-rear direction y, shear stress generated in one anti-slipping member 7 is not directly transmitted to another anti-slipping member 7 in the state where the absorbent article 1 is attached to underwear. Therefore, the position where the absorbent article 1 is attached to underwear can be prevented from displacing and the absorbent article 1 can be prevented from being distorted over a wide range. Further, by arranging a plurality of the anti-slipping members 7 in the front-rear direction y, it becomes easy to bend the absorbent article 1 in the front-rear direction y, and fittability of the absorbent article 1 to a wearer's crotch can be improved. It is preferable that each of anti-slipping members 7 aligned in the front-rear direction y has a long shape in the width direction x and is provided so as to extend continuously in the width direction x.

Preferably four or more anti-slipping members 7, more preferably eight or more those, even more preferably twelve or more those, are aligned in the front-rear direction y. The upper limit of the number of the anti-slipping member 7 is not particularly limited, and for example, 80 or less, 60 or less, 50 or less or 40 or less anti-slipping members 7 may be aligned in the front-rear direction y. The length of each of the anti-slipping member 7 in the front-rear direction y is preferably 1 mm or longer, more, more preferably 3 mm or longer, even more preferably 5 mm or longer, in view of enhancing anti-slipping effect of the anti-slipping member 7. Meanwhile, the length of each of the anti-slipping member 7 in the front-rear direction y is preferably 30 mm or shorter, more preferably 25 mm or shorter, even more preferably 20 mm or shorter, in view of facilitating smoothly bending of the absorbent article 1 in the front-rear direction y. The length of the anti-slipping member 7 in the width direction x is preferably 20 mm or longer, more preferably 25 mm or longer, and even more preferably 30 mm or longer.

It is preferable that the anti-slipping member 7 is provided so as not to extend outward in the width direction x from the absorbent body 4. That is, it is preferable that the anti-slipping member 7 is provided so as not to extend outward in the width direction x from one end and the other end of the absorbent body 4 in the width direction x. By providing the anti-slipping member 7 in this manner, slipperiness to movement of underwear at both ends of the absorbent article 1 in the width direction x is increased in attaching the absorbent article 1 to the underwear. Therefore, even when a wearer moves thighs significantly in the state where the absorbent article 1 is attached to the underwear, the absorbent article 1 is less likely to be distorted.

It is preferable that the attaching-fixing member 8 is provided so as to overlap with a front end or a rear end of the absorbent body 4, or is provided outside thereof in the front-rear direction y. Specifically, it is preferable that the attaching-fixing member 8 is provided so as to overlap with the front end of the absorbent body 4 or a front side of the front end of the absorbent body 4 in the front part 11, and the attaching-fixing member 8 is provided so as to overlap with the rear end of the absorbent body 4 or a rear side of the rear end of the absorbent body 4 in the rear part 13. By providing the attaching-fixing member 8 in this manner, the absorbent article 1 can be stably fixed to a skin facing side of underwear.

From the viewpoint of fixing the absorbent article 1 to a skin facing side of underwear more stably in attaching the absorbent article 1 to the underwear, it is preferable that the attaching-fixing member 8 is provided so as to overlap with the front end of the absorbent body 4 in the front part 11 and the attaching-fixing member 8 is provided so as to overlap with the rear end of the absorbent body 4 in the rear part 13. The absorbent article 1 tends to bend along the front end and the rear end of the absorbent body 4 due to discontinuous change in rigidity at the front end and the rear end of the absorbent body 4. However, by providing the attaching-fixing members 8 so as to overlap with the front end and the rear end of the absorbent body 4, the absorbent article 1 can be prevented from bending along the front end and the rear end of the absorbent body 4. In particular, as the front and rear end portions of the absorbent article 1 is prevented from bending in attaching the absorbent article 1 to underwear, it becomes easy to attach the absorbent article 1 to a skin facing side of underwear in a planar deployed state.

It is preferable that the attaching-fixing member 8 is wider in the width direction x than the anti-slipping member 7. Thereby, the absorbent article 1 can be stably fixed to a skin facing side of underwear. More preferably, the attaching-fixing member 8 is wider in the width direction x than the absorbent body 4.

In the front part 11 or the rear part 13, only one attaching-fixing member 8 may be provided, or a plurality of the attaching-fixing members 8 may be provided. For example, a plurality of the attaching-fixing members 8 may be aligned in the front-rear direction y in the front part 11, or a plurality of the attaching-fixing members 8 may be aligned in the front-rear direction y in the rear part 13.

The attaching-fixing member 8 provided in the front part 11 or the rear part 13 preferably has a length in the front-rear direction y of 5 mm or longer, more preferably 10 mm or longer, and preferably 80 mm or shorter, more preferably 50 mm or shorter, even more preferably 30 mm or shorter. In the case where a plurality of the attaching-fixing members 8 provided in the front part 11 or the rear part 13 are aligned in the front-rear direction y, it is preferable that the length of the entire attaching-fixing members 8 aligned in the front-rear direction y is in such a range. By providing the attaching-fixing member 8 in this manner, the position of the absorbent article 1 can be stably fixed on a skin facing side of underwear while reducing an installation area of the attaching-fixing member 8.

The anti-slipping member 7 can be formed of a resin layer. The resin layer preferably has elasticity (specifically, elasticity exerted by force that can be handled by a human hand), and is preferably composed of an elastomer resin. Examples of the elastomer resin include urethane-based resin, silicone-based resin, styrene-based resin (e.g., styrene-butadiene copolymer (SBR), styrene-isoprene-styrene block copolymer (SIS), styrene-butadiene-styrene block copolymer (SBS), styrene-ethylene-butadiene-styrene block copolymer (SEBS), styrene-ethylene-propylene-styrene block copolymer (SEPS) and the like), acrylic resin, α-olefin resin, butadiene resin, vinyl chloride resin, vinyl acetate resin, natural rubber and others.

The resin layer is preferably substantially non-adhesive, and for example, the content of a tackifier component is preferably 5% by mass or less, more preferably 3% by mass or less, even more preferably 1% by mass or less. Adhesives usually contain 10% by mass or more of a tackifier component to exhibit adhesiveness and tackiness, whereas the resin layer of the anti-slipping member 7 preferably does not contains a large amount of such a tackifier component. Examples of the tackifier component include rosin resins, terpene resins, dicyclopentadiene resins, C5 or C9 hydrocarbon resins, alicyclic hydrocarbon resins and others.

It is preferable that the anti-slipping member 7 is formed so as to rise from a surface of the non-skin facing side of the back sheet 3. The height of the anti-slipping member 7 (height from the surface of the non-skin facing side of the back sheet 3) is, for example, preferably 0.1 mm or higher, more preferably 0.2 mm or higher, even more preferably 0.3 mm or higher, and preferably 3.0 mm or lower, more preferably 2.0 mm or lower, even more preferably 1.0 mm or lower.

It is preferable that the anti-slipping member 7 has a durometer hardness A of 90 or less, more preferably 80 or less, even more preferably 70 or less, and preferably 20 or more, more preferably 25 or more, even more preferably 30 or more. Thereby, position-fixing effect or anti-slipping effect of the anti-slipping member 7 is easily exerted appropriately. The durometer hardness A is measured according to JIS K 6253-3 (2012).

Peeling adhesive strength of the anti-slipping member 7 is preferably less than 0.1 N, more preferably less than 0.05 N, and even more preferably less than 0.03 N. The peeling adhesive strength of the anti-slipping member 7 is measured according to the following method. In accordance with JIS K 6854-2 (1999) “Adhesives—Determination of peel strength of bonded assemblies (180 degree peeling)”, an integrated average load of peeling force is measured, and the average value of 10 tests is used as the peeling adhesive strength. The peeling speed (gripper movement speed) is set 300 mm/min. A test piece used for the measurement is prepared by cutting out a portion of the back sheet where the anti-slipping member is provided (a plurality of the anti-slipping members may be in alignment) in a size of 100 mm×10 mm, sticking the back sheet onto a base substrate having a size of 150 mm×25 mm, and overlaying Kanakin No. 3 cotton cloth of the same size as the base substrate on this. For the cut out back sheet, the surface opposite to the anti-slipping member is stuck onto the base substrate, and the Kanakin No. 3 cotton cloth is overlaid on the surface provided with the anti-slipping member. For the Kanakin No. 3 cotton cloth, one used for JIS L 0803 “Dyeing fastness test” is used. In the case where the size of the portion of the back sheet where the anti-slipping member is provided is less than 100 mm×10 mm, a plurality of the back sheets are joined together to form a size of 100 mm×10 mm. The back sheet provided with the anti-slipping member is stuck onto the Kanakin No. 3 cotton cloth by reciprocating a roller (manufactured by Tester Sangyo Co., Ltd., SA-1003-B, load 2 kgf, width 45 mm, diameter 95 mm) three time to prepare the test piece.

As the hook member used for the attaching-fixing member 8, a hook member of a hook-and-loop fastener can be employed, and a hook member provide with engaging parts having a shape such as a hook shape, an anchor shape or a mushroom shape is generally well known. The shape of the engaging part of the hook member is not limited to those, and the shape is not particularly limited as long as it can engage with a skin facing side of underwear.

The adhesive layer used for the attaching-fixing member 8 can be formed from a known adhesive, and a type thereof is not particularly limited. The adhesive layer preferably contains a tackifier component in an amount of 10% by mass or more, more preferably 15% by mass or more, and even more preferably 20% by mass or more. The adhesive layer has a peeling adhesive strength, that is described above, of 0.2 N or more, more preferably 0.3 N or more, and even more preferably 0.4 N or more.

From the viewpoint of enhancing shape-retaining property of the absorbent article 1, it is preferable that a fiber sheet 9 is provided between the absorbent body 4 and the back sheet 3, and the fiber sheet 9 is provided so as to extend to a front side of the front end of the absorbent body 4 and extend to a rear side of the rear end of the absorbent body 4. As a result, rigidity of the front and rear end portions of the absorbent article 1 where the absorbent body 4 does not exist can be increased. Therefore, the front and rear end portions of the absorbent article 1 can be prevented from bending, in attaching the absorbent article 1 to underwear, and the absorbent article 1 can be stably attached to a skin facing side of underwear. In view of further increasing the rigidity of the front and rear end portions of the absorbent article 1, it is preferable that the fiber sheet 9 has a larger mass per unit area than the top sheet 2 and the back sheet 3. Mass per unit area of the fiber sheet 9 is preferably, for example, 30 g/m² to 60 g/m².

It is preferable that the fiber sheet 9 extends to a front side of the attaching-fixing member 8 in the front part 11 and extends to a rear side of the attaching-fixing member 8 in the rear part 13. In particular, in the case where the attaching-fixing member 8 is provided so as to overlap with the front end or the rear end of the absorbent body 4, or is provided outside thereof in the front-rear direction y, the fiber sheet 9 is preferably provided as such. Thereby, the absorbent article 1 is less likely to bend along the front end or the rear end of the attaching-fixing member 8, and the attaching-fixing member 8 can be stably fixed to a skin facing side of underwear in attaching the absorbent article 1 to the underwear.

The absorbent article 1 is folded in three in the front-rear direction y and may be wrapped with a packaging sheet. In this case, it is preferable that the packaging sheet is overlaid on a non-skin facing side of the absorbent article 1, detachably attached to the attaching-fixing member 8, and folded in three in the front-rear direction y together with the absorbent article 1. Thereby, an individual package of the absorbent article can be formed.

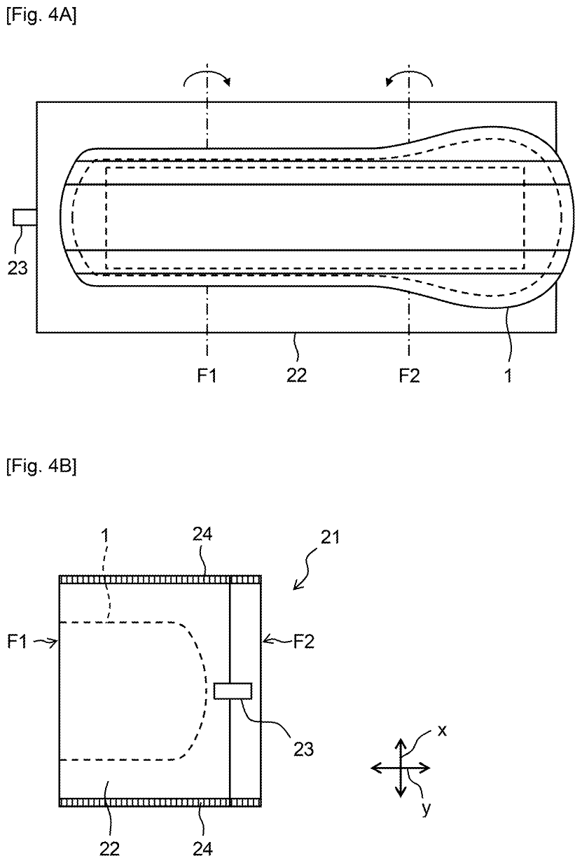

FIG. 4A and FIG. 4B show an example of a method for forming an individual package by wrapping the absorbent article shown in FIGS. 1 to 3 with a packaging sheet. FIG. 4A shows an absorbent article and a packaging sheet before wrapping, and FIG. 4B shows an individual package of the absorbent article. In FIG. 4B, the absorbent article is individually wrapped with a packaging sheet.

In an individual package 21, the absorbent article 1 is wrapped with a packaging sheet 22. In FIG. 4A and FIG. 4B, the individual package 21 is formed by folding back the absorbent article 1 together with the packaging sheet 22 at the first fold F1 and the second fold F2. Specifically, in the individual package 21, the packaging sheet 22 is overlaid on at least a part of a non-skin facing side of the absorbent article 1, the absorbent article 1 is folded back together with the packaging sheet 22 at the first fold F1 and the second fold F2 each extending in the width direction x, and as a result, the absorbent article 1 is folded in three in the front-rear direction y. At this time, the packaging sheet 22 is preferably detachably joined to the attaching-fixing member 8 provided on the back sheet 3 of the absorbent article 1.

The packaging sheet 22 may be constituted from one sheet member, or may be constituted from two or more sheet members connected to each other. The packaging sheet 22 can be composed of, for example, a plastic film, paper, a fabric material (e.g. nonwoven fabric, woven fabric, knitted fabric and the like.), or a laminate formed of any combination thereof. In the case where the attaching-fixing member 8 is made of a hook member, it is preferable that a surface of the packaging sheet 22 facing the absorbent article 1 is composed of a fabric material, and specifically, the packaging sheet 22 is preferably composed of a fabric material or a laminate whose surface facing the absorbent article 1 is made of a fabric material. In the case where the attaching-fixing member 8 is made of an adhesive layer, it is preferable that a surface of the packaging sheet 22 facing the absorbent article 1 is composed of a plastic film, and specifically, the packaging sheet 22 is preferably composed of a plastic film or a laminate whose surface facing the absorbent article 1 is made of a plastic film.

The packaging sheet 22 has an inner surface facing an absorbent article 1 side and an outer surface facing the opposite side. The inner surface and the outer surface of the packaging sheet 22 are determined based on the state where the packaging sheet 22 wraps the absorbent article 1. At least a part of the outer surface of the packaging sheet 22 is exposed to the outside of the individual package 21 and constitutes the exterior of the individual package 21.

In the individual package 21, both ends of the packaging sheet 22 in the width direction x are preferably sealed by sealing parts 24. The sealing part 24 can be formed by a known joining means such as an adhesive, heat welding, or ultrasonic welding.

It is preferable that the individual package 21 is provided with a sealing tape 23 which connects a part of the wrapping sheet 22 and another part of the wrapping sheet 22 to maintain a packaged state of the individual package 21. In FIG. 4A and FIG. 4B, the sealing tape 23 is provided so as to connect an end portion of the packaging sheet 22 in the front-rear direction y and the vicinity of the portion overlapped with the end portion in the state where the packaging sheet 22 is folded.

For taking the absorbent article 1 out of the individual package 21 to use, the folding of the individual package 21 is deployed, the packaging sheet 22 is peeled off from the absorbent article 1, and a non-skin facing side of the middle part 12 of the absorbent article 1 is set at an appropriate position on a skin facing side of underwear in the state where the absorbent article 1 is not completely deployed in a plane. Then, the absorbent article 1 is deployed in a plane with the middle part 12 of the absorbent article 1 held on the underwear, and the front part 11 and the rear part 13 are pressed against the underwear, whereby the absorbent article 1 can be appropriately attached to an intended position on the skin facing side of the underwear.

This application claims priority to Japanese Patent Application No. 2019-044010, filed on Mar. 11, 2019. All of the contents of the Japanese Patent Application No. 2019-044010, filed on Mar. 11, 2019, are incorporated by reference herein.

### REFERENCE SIGNS LIST

1: an absorbent article
2: a top sheet
3: a back sheet
4: an absorbent body
5: a side sheet
6: a rising elastic member
7: an anti-slipping member
8: an attaching-fixing member
9: a fiber sheet
11: a front part
12: a middle part
13: a rear part
21: an individual absorbent article package
22: a packaging sheet
23: a sealing tape

1. A folded absorbent article, wherein:
the absorbent article has a width direction and a front-rear direction, and comprises a top sheet, a back sheet and an absorbent body provided therebetween;
the absorbent article has a front part, a rear part and a middle part positioned therebetween in the front-rear direction, and is folded in three in the front-rear direction with a skin facing side inside so that the front part and the rear part overlap with the middle part;
the absorbent body is provided so as to extend from the middle part to the front part and the rear part;
an anti-slipping member is provided on a non-skin facing side of the back sheet in the middle part so as to overlap with the absorbent body; and
attaching-fixing members made of a hook member or an adhesive layer are provided on the non-skin facing side of the back sheet in the front part and the rear part.

2. The folded absorbent article according to claim 1, wherein
the anti-slipping member is not provided on a front side of the attaching-fixing member in the front part and on a rear side of the attaching-fixing member in the rear part.

3. The folded absorbent article according to claim 1, wherein
the attaching-fixing member is provided so as to overlap with a front end of the absorbent body or on a front side of a front end of the absorbent body in the front part, and
the attaching-fixing member is provided so as to overlap with a rear end of the absorbent body or on a rear side of a rear end of the absorbent body in the rear part.

4. The folded absorbent article according to claim 1, wherein
a fiber sheet is provided between the absorbent body and the back sheet,
the fiber sheet has a larger mass per unit area than the top sheet and the back sheet, and
the fiber sheet is provided so as to extend to a front side of a front end of the absorbent body and extend to a rear side of a rear end of the absorbent body.

5. The folded absorbent article according to claim 1, wherein
the anti-slipping member is provided only in the middle part.

6. The folded absorbent article according to claim 1, wherein
a plurality of the anti-slipping members are aligned in the front-rear direction.

7. The folded absorbent article according to claim 1, wherein
the absorbent article is folded back at a first fold line and a second fold line each extending in the width direction, thereby being folded in three in the front-rear direction, and
the anti-slipping members are provided in a region within 10 mm in the front-rear direction from the first fold line and a region within 10 mm in the front-rear direction from the second fold line.

8. The folded absorbent article according to claim 1, wherein
the anti-slipping member is formed of a resin layer.

9. The folded absorbent article according to claim 1, wherein
the anti-slipping member is provided so as not to extend outward in the width direction from the absorbent body.

10. The folded absorbent article according to claim 1, wherein
the attaching-fixing member is wider in the width direction than the anti-slipping member.

11. The folded absorbent article according to claim 1, wherein
the anti-slipping member is formed so as to rise from a surface of the non-skin facing side of the back sheet.

12. The folded absorbent article according to claim 1, wherein
the anti-slipping member has a durometer hardness A of 20 or more and 90 or less.

13. An individual absorbent article package comprising a packaging sheet and the folded absorbent article according to claim 1 wrapped with the packaging sheet, wherein
the packaging sheet is overlaid on a non-skin facing side of the absorbent article, detachably attached to the attaching-fixing member, and folded in three in the front-rear direction together with the absorbent article.
